(19) United States
(12) Patent Application Publication
(10) Pub. No.: US 2022/0088256 A1
(43) Pub. Date: Mar. 24, 2022

(54) MISTING SYSTEM AND METHOD

(71) Applicant: Luminator Holding LP, Plano, TX (US)

(72) Inventors: Michael Lee Robinson, Plano, TX (US); Joshua Cole VanderTook, Wylie, TX (US); Kirk Anthony Goins, Allen, TX (US); Daniel George Kelleher, Frisco, TX (US)

(21) Appl. No.: 17/025,919

(22) Filed: Sep. 18, 2020

(51) Int. Cl. A61L 9/14; B60H 3/00

(52) U.S. Cl. CPC A61L 9/14; B60H 3/0021; B60H 3/0035; B60H 2003/0042; A61L 2209/14; A61L 2209/16; A61L 2209/111; A61L 2209/134; B60H 2003/005

(57) ABSTRACT

A system for generating mist includes a vehicle with ventilation system, a misting device configured to atomized fluid to generate a variable amount of mist, and a fan configured to move the variable amount of mist through the ventilation system. The system further includes a particle sensor configured to measure particles and a controller in communication with the particle sensor, misting device, and fan, configured to receive a particle concentration from the particle sensor, compare it to a threshold, and control the variable amount of mist and/or the fan to keep the particle concentration greater than the threshold. A method for controlling mist in a vehicle includes pumping fluid to a misting device, generating a mist of fluid, pumping the mist into a vehicle ventilation system, measuring a particle concentration, and manipulating a misting device, pump, and/or fan using a controller to keep the particle concentration above a threshold.

### TECHNICAL FIELD

This disclosure relates to dispensing mist and more particularly, to dispensing mist in a space.

### BACKGROUND

Cleaning the surfaces inside vehicles and other spaces is difficult and time consuming and often includes using manual labor and various hand tools. Cleaning to a point of disinfection of surfaces is even more difficult and time consuming and traditionally cannot be done during operation of a vehicle or when a space is being actively used. Handheld disinfecting devices that use an atomized spray are used to disinfect surfaces, but they cannot disinfect air supply or ventilation systems easily. The atomized spray of disinfectant used by disinfecting devices needs to have a high enough concentration so that the disinfectant works properly. However, the concentration decreases with the distance that the atomized spray travels. Additionally, handheld disinfecting device commonly use chemicals which are harmful to the respiratory systems of humans in various concentrations. Disinfecting devices also cannot precisely regulate the concentration of the atomized spray of disinfectant in the air, usually using on/off or user-controlled atomization spray rates.

### SUMMARY

A system for generating and controlling a mist in a vehicle is provided. The system includes a vehicle comprising a ventilation system with outlets distributed within the vehicle and a supply of fluid. The system further includes a misting device configured to atomize an amount of fluid to generate a variable amount of mist in air with the mist having a first range of particle sizes. The system also includes a fan having a variable speed and configured to move the mist through the ventilation system and out of the outlets. A particle sensor is also included which is configured to measure a quantity and size of particles in air with the particle sensor being located to measure the quantity and size of particles in the air in the vehicle. The system further includes a controller in communication with the particle sensor with the controller in communication with and controlling at least one of the variable amount of mist in the air, or the variable speed of the fan. The controller is configured to receive the quantity and size of particles in the air in the vehicle. The controller is further configured to determine a particle concentration in the air in the vehicle based on the received quantity and size of particles, of particles in a second range of particle sizes. The second range of particles sizes overlap with the first range of particle sizes. The controller is also configured to compare the particle concentration to a threshold concentration and control at least one of the variable amount of mist in the air or the variable speed of the fan in order to have the particle sensor measure a particle concentration greater than the threshold concentration.

A system for generating and controlling a mist in a vehicle is also provided. The system comprises a vehicle with the vehicle comprising a ventilation system with outlets distributed within the vehicle. The system also includes a supply of fluid and a misting device configured to atomize an amount of fluid to generate a variable amount of mist in air. The system further includes a fan having a variable speed and configured to move the mist through the ventilation system and out of the outlets. A particle sensor is also included which is configured to measure a concentration of atomized particles within two or more size ranges of particles with the particle sensor being located to measure the concentration of atomized particles within two or more size ranges of particles in the vehicle. The system also comprises a controller in communication with the particle sensor with the controller in communication with and controlling at least one of the variable amount of mist in the air or the variable speed of the fan. The controller is configured to receive the concentration of atomized particles within two or more size ranges of particles from the particle sensor. The controller is further configured to determine a concentration of atomized particles having sizes within at least one of the two or more size ranges of particles and compare the concentration of atomized particles to a threshold concentration. The controller is also configured to control at least one of the variable amount of mist in the air or the variable speed of the fan in order to have the particle sensor measure a concentration greater than the threshold concentration.

A method for generating and controlling a mist in a vehicle is also provided. The method includes pumping a fluid from a fluid reservoir to a misting device using a pump. The method also includes generating a mist of the fluid at a first point using the misting device such that the mist of the fluid is generated within air. The method further includes pumping the mist of the fluid into a vehicle ventilation system using a fan and measuring a particle concentration of the mist of the fluid within the air at a second point using a particle sensor with the second point being external to the vehicle ventilation system. The method also includes manipulating at least one of the misting device, the pump, or the fan using a controller such that the particle concentration of the mist of fluid within the air is greater than a threshold.

### BRIEF DESCRIPTION OF DRAWINGS

FIG. 1A-1C are example perspective views of vehicles which include a system for generating and controlling mist in the vehicles according to an aspect of the present disclosure.

FIG. 2 is an example diagrammatic view of a system for generating and controlling mist in a vehicle according to an aspect of the present disclosure.

FIG. 3 is an example perspective view of a vehicle ventilation system according to an aspect of the present disclosure.

FIG. 4 is an example perspective view of a misting device according to an aspect of the present disclosure.

FIG. 5 is an example diagrammatic view of a particle sensor according to an aspect of the present disclosure.

FIG. 6 is an example perspective view of a misting system according to an aspect of the present disclosure.

FIG. 7 is an example schematic view of a control of a misting system according to an aspect of the present disclosure.

FIG. 8 is an example flow diagram of a method of misting according to an aspect of the present disclosure.

FIG. 9 is an example flow diagram of a method of misting according to an aspect of the present disclosure.

### DETAILED DESCRIPTION

The following detailed description is exemplary in nature and is not intended to limit the scope, applicability, or configuration of the invention in any way. Rather, the following description provides some practical illustrations for implementing examples of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives.

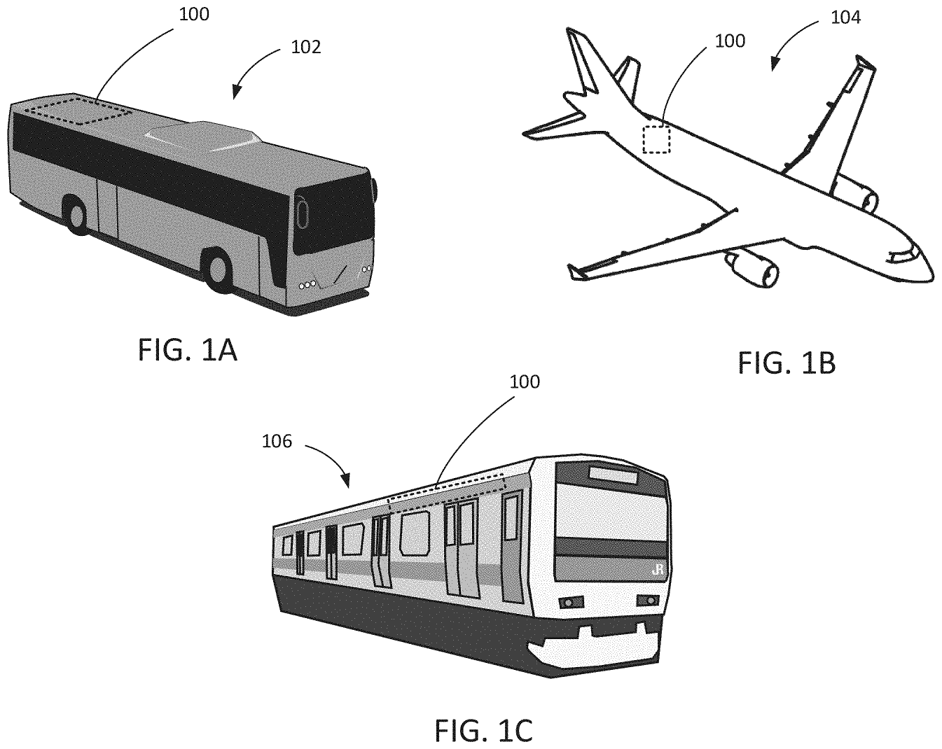

FIG. 1A-1C are example perspective views of various vehicles which include a system for generating and controlling mist 100 in the vehicle according to an aspect of the present disclosure. The vehicle can be any type of vehicle including motor vehicles such as a bus 102. Other vehicles are also contemplated which include a system for generating and controlling mist such as an airplane 104 and train 106. Other vehicles which can include a system for generating and controlling mist are also contemplated and can include trucks, cars, and boats. The system for generating and controlling mist in the vehicle can include components located within the interior of the vehicle and components located on the exterior of the vehicle. In some examples, all components are located within the vehicle. In some examples, some components of the system can be located remotely from the vehicle as is described later herein.

The system for generating and controlling mist is not limited to vehicles. In some examples, the system for generating and controlling mist can be used in any enclosed space including rooms, elevators, bathrooms, and theaters. In some such examples, the system can be in communication with a heating ventilation air conditioning (HVAC) system which can circulate air within an enclosed space. In some such examples, the system for generating and controlling mist can be added to an existing HVAC system such that mist is circulated using the HVAC system and the HVAC system is controlled to maintain a desired particle concentration within the enclosed space. In some other examples, the system for generating and controlling mist can be integrated with an HVAC system, existing or new, to maintain a desired particle concentration within the enclosed space. In some examples, the enclosed space can include doors or other openings to non-enclosed spaces.

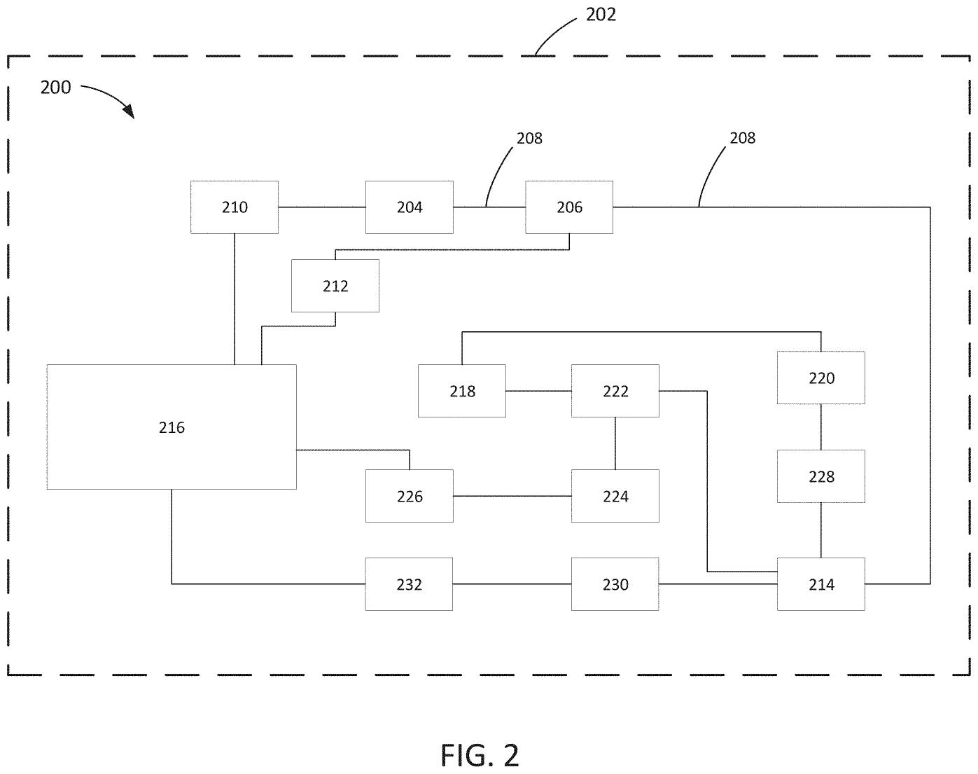

Turning to FIG. 2, FIG. 2 is an example diagrammatic view of a system for generating and controlling mist 200 in a vehicle 202 according to an aspect of the present disclosure. The system includes a vehicle 202 which houses a fluid reservoir 204, a pump 206, tubing 208, a fluid level detection circuit 210, a pump circuit 212, and a misting device 214. Fluid reservoir 204 can contain a fluid, which can be any type of fluid, and can be considered a supply of fluid. In some examples, the fluid reservoir 204 contains a fluid which has anti-bacterial or anti-viral properties. In some examples, the fluid is a disinfectant. Such fluid can inhibit propagation of bacteria and/or viruses in a space in addition to destroying bacteria and viruses. The fluid can also inactivate viruses and/or bacteria. In some examples, the fluid is a mixture of fluids such as an anti-viral fluid and water. In some examples, the fluid can be non-toxic to humans and in further examples, the fluid is non-toxic to human inhalation when atomized in a mist. Fluids which fluid reservoir 204 contains can also include fluids with antiseptic, antifungal, and/or sterilizing properties. In some examples, the fluid is triethylene glycol. A person having ordinary skill in the art will recognize other fluids are contemplated.

In the example of FIG. 2, fluid reservoir 204 is connected to fluid level detection circuit 210. Fluid level detection circuit 210 can detect the amount of fluid within fluid reservoir 204. In some examples, the fluid level detection circuit 210 is connected to a float which sits inside of fluid reservoir 204. This float can float on top of the fluid located within fluid reservoir 204 and determine how much fluid is within fluid reservoir 204 by communicating its position to fluid level detection circuit 210. Fluid level detection circuit 210 can interface with a controller 216 and provide information about the amount of fluid remaining within fluid reservoir 204. In some examples, fluid level detection circuit 210 can provide an alert when the fluid level is low in the fluid reservoir 204. In such examples, a user can be prompted to add fluid to fluid reservoir 204.

In FIG. 2, fluid reservoir 204 is also connected to pump 206. Fluid reservoir 204 and pump 206 can be fluidly connected using tubing 208. In some examples, fluid reservoir 204 is directly connected to pump 206 such that no tubing is used. Pump 206 can pump fluid such that the fluid is transported from fluid reservoir 204 through tubing 208 and through pump 206. In some examples, fluid reservoir can be pressurized by a pump such that fluid flows from fluid reservoir 204 directly to misting device 214 though tubing. In some examples, pump 206 is an inline pump and, in the example of FIG. 2, pump 206 is a peristaltic pump. Using a peristaltic pump can be advantageous as the components of the pump 206 do not touch the fluid being pumped. Thus, the fluid being pumped is uncontaminated by any contaminants located within the pump. However, in some examples, the pump is a fluid pump in which fluid is pumped from the reservoir, through the tubing, and through the pump, wherein the fluid comes in contact with the components of the pump. Other pumps are contemplated and can be used such as gear pumps, diaphragm pumps, and centrifugal pumps etc.

Further in the example of FIG. 2, pump 206 is connected to misting device 214 through tubing 208. Tubing 208 allows fluid to pass through and fluidly connects various parts of the system which use fluid (e.g. fluid reservoir 204 and pump 206). In some examples, other fluid connectors are used such as hoses or pipes. In operation, pump 206 pumps fluid stored in fluid reservoir 204 through tubing 208 to misting device 214. In FIG. 2, pump 206 is connected to pump circuit 212 which can control aspects of the pump 206. Some aspects of the pump 206 which pump circuit 212 can control are whether the pump is on or off, the flow rate, the speed of the pump's motor, the pressure of the fluid, and activating valves to be open or closed. Other controllable aspects of pump 206 are contemplated. By controlling various aspects of the pump 206, pump circuit 212 can provide specific amounts of fluid at specific pressures to misting device 214. In FIG. 2, pump circuit 212 is additionally connected to controller 216 such that the pump circuit 212 provides information to the controller 216 about the various aspects of the pump (e.g. flow rate) and such that controller 216 can control the pump circuit 212.

Continuing with the example of FIG. 2, the system for generating and controlling mist in a vehicle includes an air inlet 218, an air outlet 220, and an optional air filter 222. Air inlet 218 can receive air from inside or outside of vehicle 202 and the air can pass through air filter 222. In some examples, a blower motor is used to intake air from the inside or outside of the vehicle and into air inlet 218. In some examples, the blower motor is a fan or turbine. As air passes through air filter 222, contaminants can be removed. In some examples, air filter 222 is a high efficiency particulate air (HEPA) filter which can remove particulates from the air. Using a HEPA filter can be advantageous over a non-HEPA filter as a HEPA filter can remove more particulates which can be harmful to humans. In some examples, more than one air filter can be used to further decrease the number of particulates/contaminants within the air.

After air passes through the air filter 222, it can then optionally be treated by ultraviolet light (UV) so that the air is further decontaminated. In the example of FIG. 2, a UV lamp 224 is used to provide the UV light such that air surrounding the UV lamp is treated by the UV light. The UV lamp 224 can be powered by a UV lamp driver 226. In some examples, more than one UV lamp can be used. In some examples, UV light emitting diodes (LEDs) are used to provide the UV light. Using UV light can be advantageous as it can decontaminate the air, destroying microorganisms such as bacteria, fungi, and viruses which may be present in the air. In some examples, only air filter 222 is used to decontaminate the air, however, in some examples, air filter 222 supplemented with a UV lamp 224 before and/or after the air filter 222. In some examples, no air filter is used and only UV light from a UV lamp is used to decontaminate the air. However, by using both UV light from a UV lamp and an air filter, the number of contaminants in the air can be reduced further than if only one of the UV light from a UV lamp and the air filter are used. In some examples, no air filter or UV light is provided and air flows directly through air inlet. In FIG. 2, after passing the UV lamp 224, the air the passes into misting device 214 and/or into the area misting device 214 is active and further passes through air outlet 220 via ducts 228. Air outlet 220 can lead into the interior of the vehicle where passengers are located (e.g. cabin). In some examples, air outlet 220 is a diffuser. In some examples, air outlet 220 leads to further outlets such as adjustable air nozzles in a cabin of a vehicle. Further, in some examples, air outlet 220 can be multiple outlets and in some examples, can be outlets within a vehicle ventilation system.

In some examples, air inlet 218, air outlet 220, and air filter 222 are located within a vehicle ventilation system. The vehicle ventilation system can have a separate air inlet, air outlet, and air filter which can be connected to the air inlet 218, air outlet 220, and air filter 222 of the system for generating and controlling mist in the vehicle. In some such examples, the air inlet of the vehicle ventilation system can intake air from the interior and/or exterior of the vehicle and pass it through a filter before air reaches the air inlet 218 of FIG. 2. For example, air inlet 218 can intake air from the passenger cabin where air outlet 220 has put air into (e.g. recycled air). Further, in some examples, the air outlet of vehicle ventilation system exhausts air from the inside of the vehicle to the outside of vehicle or in some examples, exhausts air from the inside of the vehicle into air inlet 218. It can be advantageous to have a separate vehicle ventilation system which can be connected to the system of FIG. 2 as the separate vehicle ventilation system can be used independently of, or in conjunction with, the system of FIG. 2. For example, if the system of FIG. 2 is inoperable, the vehicle ventilation system can still provide air to the interior of the vehicle. In addition, vehicles which already have a vehicle ventilation system may require less modification of the system to incorporate the system of FIG. 2.

Continuing with the example of FIG. 2, a fan 230 is connected to misting device 214 and can blow the mist to air outlet 220. Fan 230 and any other fans in the vehicle ventilation system can be controlled by a fan speed control circuit 232 such that they blow a specific amount of generated mist and/or air through the vehicle ventilation system. The fan speed control circuit 232 can control one or more aspects of the fan 230 such as the speed, duty cycle, and cubic feet per minute (CFM) output. In some examples, fan 230 is a variable speed fan. The fan speed control circuit 232 can be further connected to controller 216 and/or to sensors (e.g. airflow sensor) which can provide input to the fan speed control circuit 232. The fan speed control circuit 232 can receive the input from the controller 216 and/or sensors and use the input to control the aspects of the fan (e.g. speed).

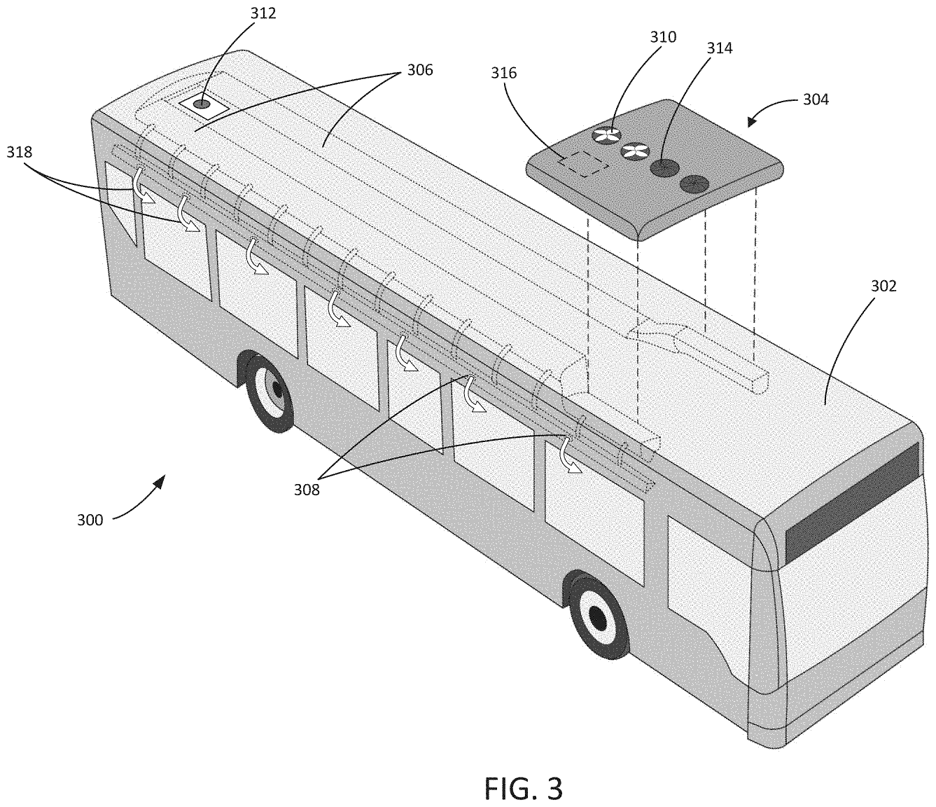

Moving to the example of FIG. 3, FIG. 3 is an example perspective view of a vehicle ventilation system according to an aspect of the present disclosure. In the example of FIG. 3, vehicle ventilation system 300 of vehicle 302 can include a rooftop unit 304, ducts 306, vents/outlets 308, fans 310, and exhaust outlet 312. Rooftop unit 304 can have air inlets 314 with fans 310 that bring in air from outside the vehicle to the inside of the vehicle and can additionally have filters which remove contaminants from the air. The rooftop unit 304 can be connected to one or more ducts 306 which direct the air brought in by the rooftop unit 304 inside of the vehicle. The ducts 306 can include various connections, vents, outlets, diffusers, or the like distributed within the vehicle to direct the air from within the ducts to locations inside the cabin of the vehicle. In the example of FIG. 3, ducts 306 are connected to vents/outlets 308. When in operation, the vehicle ventilation system of FIG. 3 intakes air from outside of the vehicle using the rooftop unit 304, passes the air through a filter 316 passes the air through ducts 306, optionally using fans 310 to push the air through the ducts 306, passes the air through the connections and vents/outlets 308 in the cabin of the vehicle (e.g. 318), and finally exhausts the air outside of vehicle 302 through exhaust outlet 312 or optionally, back into the vehicle 302 through ducts 306. In some examples, the rooftop unit 304 draws in substantially all air from the outside of the vehicle while in other examples, the rooftop unit 304 draws in substantially all air from inside the vehicle. In further examples, some air is drawn from outside of the vehicle and some air is drawn from inside the vehicle. While the vehicle ventilation system of FIG. 3 is described, other vehicle ventilation systems are contemplated. For example, aerial vehicle ventilation systems which can include different components for intaking and distributing air within the cabin are contemplated and a person having ordinary skill will recognize that the present disclosure is not limited to ventilation systems as described in the example of FIG. 3.

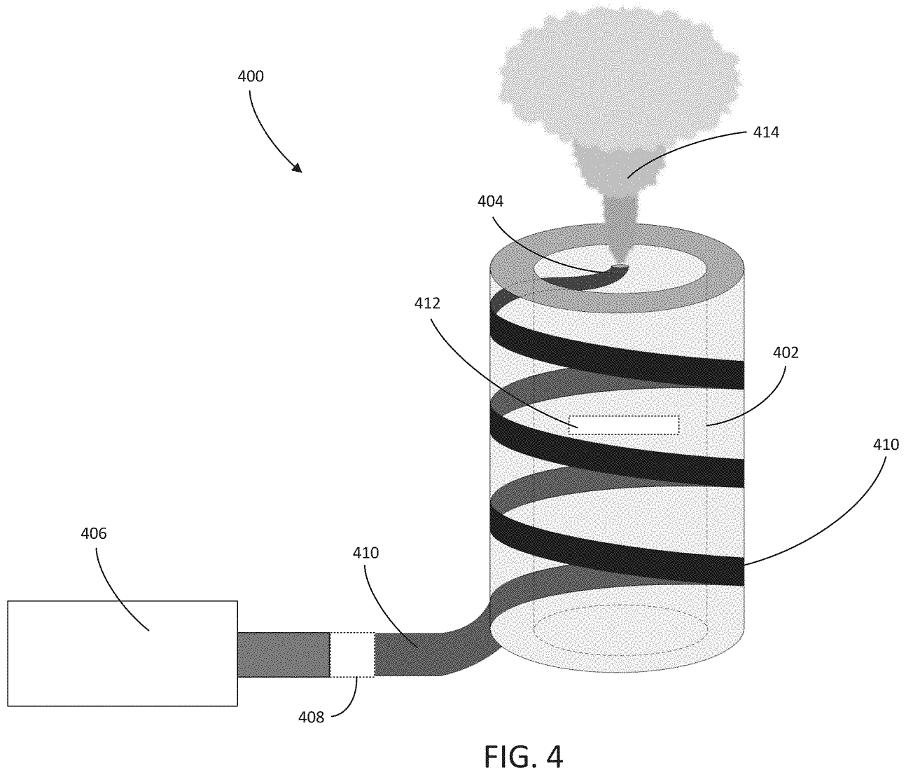

Moving to FIG. 4, FIG. 4 is an example misting device according to an aspect of the present disclosure. In the example of FIG. 4, misting device 400 comprises a heating element 402 and a nozzle 404 for atomizing a fluid. In some examples, heating element 402 is a heat exchanger. In some examples, heating element 402 is separate from misting device 400. Fluid that is atomized can be in the form of a fog, mist, spray, haze, cloud, vapor, aerosol, droplets or other forms of fluid being in the air (e.g. 414). Misting device 400 is fluidly connected to a fluid reservoir 406 and a pump 408 via fluid line 410 which can pump fluid to misting device 400 from fluid reservoir 406. Heating element 402 can heat up fluid in fluid line 410 which has been pumped into the misting device 400 such that the fluid atomizes into fluid droplets in the air (e.g. into a mist). In FIG. 4, the fluid line 410 is coiled around heating element 402, but other configurations of fluid line 410 and heating element 402 are contemplated. For example, heating element 402 can be coiled around a fluid line 410. Atomized fluid can be expelled from the misting device 400 through nozzle 404. In some examples, misting device 400 generates a variable amount of mist 414 in the air. In some examples, the nozzle atomizes the fluid into mist 414 without using heating element 402.

In the example of FIG. 4, the vaporized fluid is expelled from the misting device 400 using a fan 412. Fan 412 can blow air past nozzle 404 so that the mist generated at the nozzle 404 is disbursed in a desired manner (e.g. to a vehicle ventilation system). Fan 412 can be located in a location proximate to nozzle 404 which includes in front of nozzle 404, behind nozzle 404, and to the side of nozzle 404. More than one fan can be used and in some examples no fan is used. However, it is advantageous to include at least one fan such that the generated mist does not immediately condense near the fluid nozzle. In some examples, the vaporized fluid is expelled from the misting device 400 due to a pressure difference between the air and the vaporized fluid. In some examples, misting device 400 can be a pressurized mister.

Misting device 400 can be connected to ducts, vents, air outlets or other structures which can transport the atomized fluid away from the misting device to a desired location (e.g. vehicle ventilation system 300 of FIG. 3). In operation, misting device 400 can generate a mist of atomized fluid into a vehicle ventilation system such that the mist is dispensed throughout the vehicle. Fan 412 can help direct the mist of atomized fluid from misting device 400 through the vehicle ventilation system and out of any vents or outlets, though in some examples, one or more additional fans are used to direct the mist of atomized fluid through the vehicle ventilation system and out of the outlets.

In some examples, the misting device 400 includes one or more nozzles and a pump. The pump 408 can be the same pump or a different pump than the pump which pumps fluid from the fluid reservoir to the misting device. The pump can pump a fluid through the one or more nozzles at a pressure which can cause the fluid to atomize. In some examples, the pump 408 can pump air in addition to or in lieu of fluid such that fluid supplied to the misting device is atomized. In some examples, both a heating element and a pump providing pressure to one or more nozzles are used to atomize fluid into a mist. In some examples, the misting device can be substantially enclosed such that mist is only directed through a desired opening.

In some examples, the misting device is an ultrasonic misting device. In some such examples, a disc is submersed in a fluid and is vibrated at a frequency which causes the fluid to be suspended in the air, thereby generating a mist. In some examples, the disc generates the most mist when submersed in the fluid at a specific level. In such examples, a pump and a sensor are used to detect and ensure that the specific level of fluid is maintained such that the disc generates mist most efficiently. A person of ordinary skill will understand that other misting devices which atomize a fluid are contemplated and that more than one misting device can be used of any type of misting device.

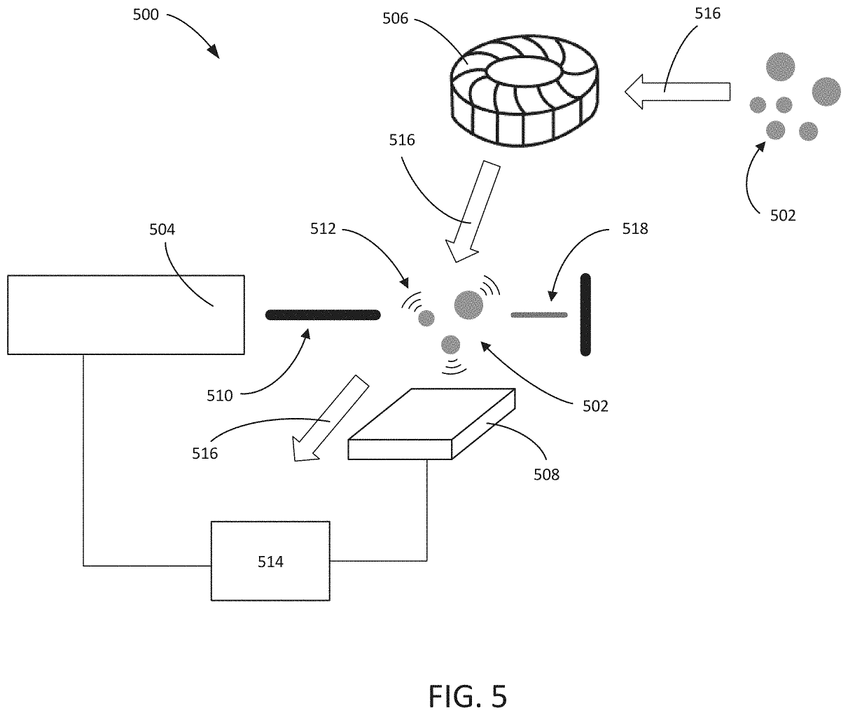

Moving to FIG. 5, FIG. 5 is a diagrammatic view of a particle sensor according to an aspect of the present disclosure. Particle sensor 500 can detect the number and/or the size of particles 502 within the air it samples. In some examples, particle sensor can detect the concentration of particles within the air it samples. Particles 502 can include any matter in the air including atomized fluid, fog, mist, spray, haze, cloud, vapor, aerosol, droplets or other forms of matter in the air. In FIG. 5, particle sensor 500 is a laser particle sensor and comprises a laser 504, a fan 506, and at least one optical sensor 508. In operation, particle sensor 500 energizes the laser 504 which can have a portion of the laser beam 510 scatter from hitting particles 502. The scattered light 512 can then be measured by the at least one optical sensor 508 which can determine the properties of the reflected light (e.g. intensity). In some examples, transmitted light 518 is also measured. The measurement(s) can then be fed to a controller 514 which can determine the size of particles which scattered the light of the laser 504. Each particle detected can be recorded such that a total number of particles can be detected by the laser particle detector. The particle sensor of FIG. 5 can detect particles ranging in size from 0.3 microns in diameter to 2.5 microns in diameter. In some examples, the range can be larger or smaller than 0.3 microns to 2.5 microns (e.g. 0.1 microns to 10 microns). In some examples, particle sensor 500 can be used to detect particles which have a specific size which can correlated with a specific type of particle. It can be advantageous to detect the size of particles in addition to the number of particles in order to measure the concentration of a specific compound in the air (e.g. mist of fluid) and ignore other particles such as dust or water vaper.

Particle sensor 500 includes a fan 506 which blows air 516 through the particle sensor 500 such that the laser 504, one or more optical sensors 508, and controller 514 can repeat the process of determining the number and/or size of particles over time and can thereby detect the number of particles of a specific size. The fan 506 can be controlled by the controller 514 such that a known amount of air is passed through the particle sensor 500, aiding in the calculation of the number and/or size of particles detected by particle sensor 500. In some example, particle sensor 500 can determine the number of particles 502 and the size of particles 502 substantially simultaneously. Particle sensor 500 can be configured to measure/sample the air continuously or at discrete points over time. Continuously can be considered multiple times a second and includes thousands of times per second. By sampling continuously, particle sensor 500 can determine the number of particles 502 in the air (e.g. concentration) at any time. By measuring/sampling at discrete points in time, particle sensor 500 can determine the number of particles 502 in the air at specific times. Continuous measuring/sampling can be more accurate at determining a change in the number of particles in the air, however, discrete measuring/samples can reduce the power consumption and amount of data generated by particle sensor 500. In some examples, particle sensor 500 can be configured to measure in response to inputs from other sensors or controllers and in some examples, particle sensor 500 outputs any measurements to a separate controller (e.g. 216 of FIG. 2).

In some examples, particle sensor 500 can determine the number of particles within one range of sizes and the number of particles in a different range of sizes substantially simultaneously. In some examples, particle sensor 500 can be an aerosol particle counter which can count and size the number of particles in the air. In some examples, particle sensor uses laser absorption to determine the number and/or size of particles within the air. While a laser particle sensor has been described, particle sensor 500 can be any type of particle sensor. In some examples, the type of particle sensor can measure a number and/or size of particles. Some examples include an infrared emitter sensor, a condensation particle counter, and direct imaging sensor. In some examples, the particle sensor can measure a concentration of particles within the volume. For example, the particle sensor can measure a parts per million (ppm) concentration of particles in the air. In some further examples, the particle sensor can measure a mass concentration. In some examples, the particle sensor can measure any combination of particle count, particle size, concentration, and mass concentration.

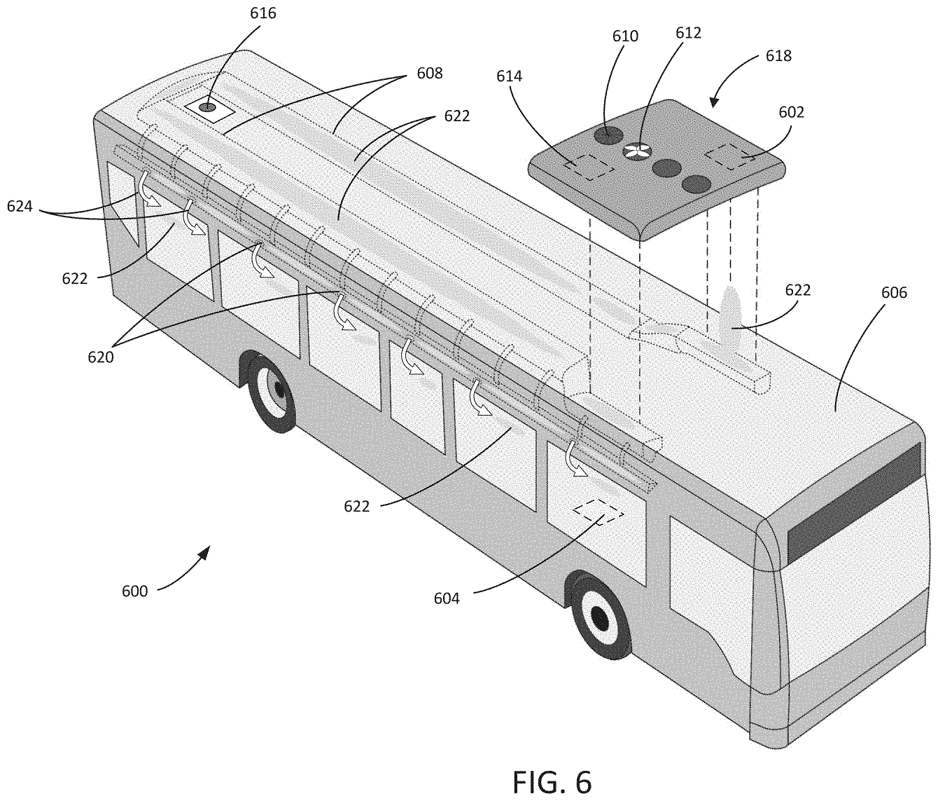

Moving to FIG. 6, FIG. 6 is a perspective view of an example misting system according to an aspect of the disclosure. Misting system 600 includes a misting device 602 and a particle sensor 604 located within a vehicle 606. As discussed above, particle sensor 604 can detect the size and/or number of particles in the air. In FIG. 6, particle sensor 604 is configured to detect the size and number of particles which correspond with the particles of fluid that are generated into a mist by misting device 602. Particle sensor 604 can detect the number of fluid particles in the air (e.g. concentration) by sampling the air over time. In FIG. 6, particle sensor 604 is located inside of vehicle 606 at a position in which it can measure the quantity and size of atomized particles from misting device 602. In some examples, particle sensor is located within a vehicle ventilation system which can include ducts 608, air inlets 610, fans 612, air filter 614, outlet exhaust 616, and rooftop unit 618. In some examples, particle sensor is located external to vehicle ventilation system near outlets/vents 620. Placing particle sensor near outlets/vents 620 can be advantageous because the air delivered by outlets/vents 620 can be the closest to passengers in the vehicle 606. Further, in some examples, particle sensor 604 can be located near an entrance or exit of a vehicle. In such examples, the particle sensor can detect if a passenger has opened a door, which could cause the amount of mist in the air to change, and relay that information to misting device 602. In some examples, more than one particle sensor is used. When more than one particle sensor is used, the sensors can be used in different locations throughout the interior of the vehicle 606. This can be advantageous as the concentration of fluid in the air can vary throughout the vehicle. In some examples, multiple particle sensors are used at various heights within a vehicle (e.g. head level and floor level). This can be advantageous as in some examples, a vehicle ventilation system can change the amount of fluid in the air at different heights by using vents. A person having ordinary skill will appreciate that other positions of particle sensor 604 are contemplated and that this disclosure is not limited to any number of particle sensors.

Continuing with FIG. 6, mist 622 generated by misting device 602, is pumped throughout the ducts 608 of the vehicle ventilation system to outlets/vents 620 and into the interior of vehicle 606 where passengers can be located. As shown by the arrows 624, the flow of air from the ducts carries the mist of fluid throughout the vehicle 606.

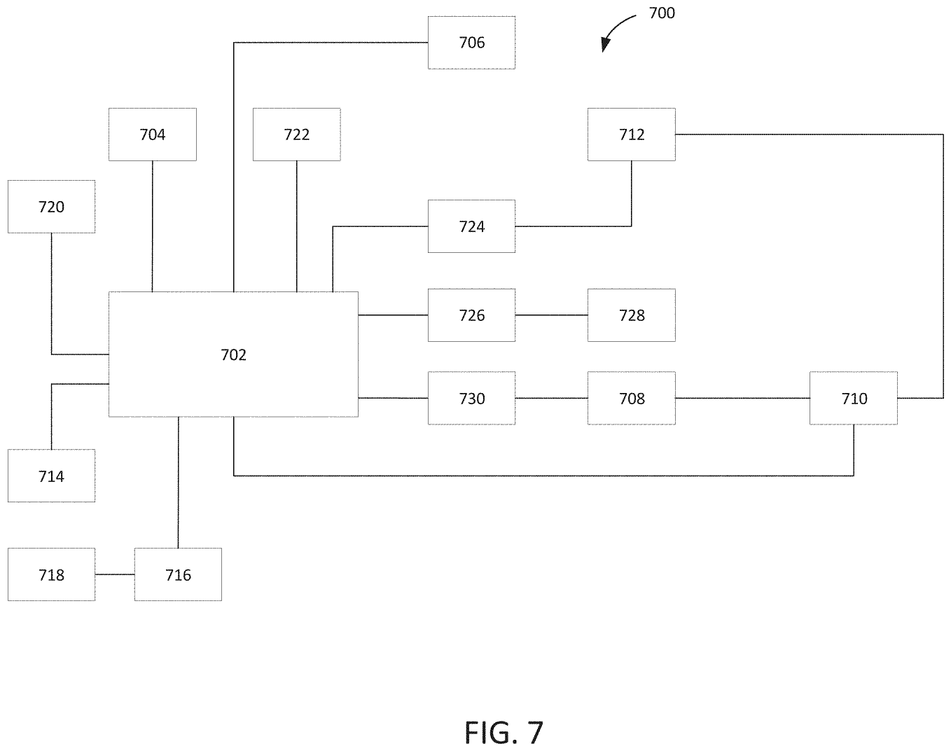

Moving to FIG. 7, FIG. 7 is a schematic view of a control of a misting system 700 according to an aspect of the present disclosure. Controller 702 can be implemented as a microcontroller (e.g. ASIC, FPGA) and/or as a processor (e.g. microprocessor). In some examples, controller 702 can include and/or be in communication with a computer readable medium 704, for example, non-volatile memory, which can store data including computer code. In some examples, controller 702 can be multiple controllers in communication with each other (e.g. a particle sensor controller and a separate controller). In the example of FIG. 7, controller 702 is in communication with particle sensor 706, fan 708, misting device 710, and pump 712. Controller 702 is further in communication with vehicle network 714, power supply regulator 716, power supply 718, visual status indicator 720, fluid level detection circuit 722, pump circuit 724, UV lamp driver 726, UV lamp 728, and fan speed control circuit 730. A person of ordinary skill will appreciate controller 702 can be in communication with other devices, circuits, and sensors and the present disclosure is not limited by connections to controller 702. In some examples, other sensors can be in electronic communication with controller 702 such as temperature sensors, barometric sensors, humidity sensors, fluid reservoir level sensors, sensors for detecting the volume of fluid pumped by one or more pumps, and other sensors. These other sensors can provide data to controller 702 such that controller 702 can determine how the misting system should be operated. For example, a sensor which detects the opening of a door of a vehicle can cause controller 702 to increase an output of the misting system 700 in order to adjust to the resulting decrease in fluid concentration within the vehicle. In some examples, a sensor which detects the dispensing volume of the fluid provides input into the controller such that the controller can determine if the dispensing volume is at a desired level. In some further examples, a visible light or infrared camera can be used to provide data to controller 702. In some such examples, the camera can detect the number of people in an area or on a vehicle and the controller can adjust the output of the misting system 700 based on the number of people present in the area. In some examples, a infrared camera can detect if a person boarding a vehicle has an elevated temperature and the controller 702 can control misting device 710 can increase the amount of mist generated. A person having ordinary skill in the art will recognize other sensors which can be in communication with the controller are contemplated. Further, in some examples, controller 702 can receive any input in addition to any measurement of particles from particle sensor 706 to manipulate the amount of mist generated and/or already in the air. Inputs can be from sensors, cameras, manual inputs from users (e.g. buttons), remote inputs, signals, vehicle systems, or from other things. A person having ordinary skill will appreciate that any input can be used by controller 702 to adjust the aspects of misting system 700.

In the example of FIG. 7, controller 702 can receive data from particle sensor 706 which can include a quantity and/or size of particles in the air. Additionally, controller 702 can receive data from the fan 708 (e.g. rpm of the fan), misting device 710 (e.g. duty cycle), the heating element of misting device (e.g. temperature), and pump 712 (e.g. flow rate). This data can be used by controller 702 to determine how to manipulate the fan 708, misting device 710, the heating element of misting device 710, and/or pump 712, such that a desired amount of fluid is atomized by misting device 710. In operation, particle sensor 706 can measure a first quantity and/or size of particles in the air which can correlate to a first concentration of fluid in the air. The measurement of the first quantity and/or size of particles can be sent to controller 702 electronically. Controller 702 can use the first quantity and/or size of particles to determine if the first concentration of fluid in the air is at, above, or below a desired threshold by comparing the first quantity to the threshold. The desired threshold can be a range of concentrations or a single concentration. In some examples, the desired threshold is a range or point at which the concentration of fluid in the air is safe for human inhalation and effective at disinfecting. In some examples, the desired threshold is a range or point at which the concentration of fluid in the air is most effective at disinfecting. It can be advantageous to have a concentration that is most effective at disinfecting as people are not always present in vehicles when disinfection is desired. Controller 702 can manipulate fan 708, misting device 710, the heating element of misting device 710, and/or pump 712 to increase/decrease/maintain the concentration of fluid in the air to reach the desired threshold. In some examples, controller 702 can directly control the fan 708, misting device 710, the heating element of misting device 710, and/or pump 712. However, in some examples, controller 702 can indirectly control the fan 708, misting device 710, and/or pump 712 such as through fan speed control circuit 730, and pump circuit 724. In some examples, controller 702 can individually control the fan 708, misting device 710, the heating element of misting device 710, and pump 712. Alternatively, in some examples, controller 702 controls the fan 708, misting device 710, the heating element of misting device 710, and pump 712 all together or in various combinations. For example, controller could control fan 708 and misting device 710 as a single combination and separately, the control pump 712 and the heating element of misting device 710.

In examples in which the first concentration of fluid in the air is below the desired threshold, controller 702 can communicate with the fan 708, misting device 710, the heating element of misting device 710, and/or the pump 712. The controller can communicate to the misting device 710 to increase an output of mist generated. Increasing the output of mist generated can include increasing the amount of time the misting device 710 is generating mist in a period of time (e.g. increasing duty cycle). The controller can also communicate with the pump 712 of misting device 710 to increase the amount of fluid delivered to misting device 710, whereby the increased amount of fluid delivered to misting device 710 increases the amount of fluid generated into a mist by misting device 710. Additionally, in such examples, the heating element of misting device 710 can increase its heat output, thereby increasing the amount of fluid atomized by misting device 710. Further, in such examples, the controller can increase the fan speed and/or duty cycle of fan 708 to move the increased amount of mist generated by misting device 710 into the air. The controller can continue this operation, thereby increasing the concentration of fluid in the air, until particle sensor 706 measures the concentration of fluid to be the desired concentration (e.g. at or above the desired threshold) and communicates as such to controller 702. Controller 702 can again manipulate fan 708, misting device 710, the heating element of misting device 710, and/or pump 712 such that the concentration of fluid in the air remains at, or above the desired threshold.

The configuration of FIG. 7 can be advantageous because it is a closed loop system. In such systems, mist generation by the misting system is controlled without manual input. For example, in FIG. 7, the controller 702 uses the information from particle sensor 706 to determine if the misting system should increase the amount of mist generated, decrease the amount of mist generated, or keep generating the same amount of mist. A closed loop system can be advantageous to an open loop system as the closed loop system can sense the output and adjust the input accordingly.

Continuing with FIG. 7, controller 702 can be in communication with a vehicle network 714. Vehicle network 714 can include processors, controllers, sensors, networking devices, imaging devices and other devices which interface with aspects of the vehicle. For example, vehicle network 714 can include a vehicle bus to which various modules are connected. In some examples, vehicle network 714 can include one or more processors in electronic communication with sensors and networking devices such that the control of the vehicle network 714 can be done remotely. In some examples, vehicle network 714 can send and receive data with remote devices. For example, the vehicle network can be a plane network which can send and receive data from satellites. In some examples, vehicle network 714 and the misting system can be monitored and/or controlled entirely remotely. In some examples, vehicle network 714 can control all or a portion of the misting system and in some examples, vehicle network includes sensors which can provide input to misting system to increase, decrease, or maintain a concentration of fluid in the air. Vehicle network 714 can further include data storage (e.g. non-volatile memory) which can record various aspects of the vehicle including the misting system. For example, vehicle network 714 can record the status of the misting system, whether it was active or inactive, the various settings of the fan, misting device, and pump, and the measurements of the particle sensor.

In the example of FIG. 7, the misting system further includes connections to a power supply 718 (e.g. vehicle battery) and a power supply regulation device 716. The connections to the power supply can connect the parts of the misting system 700 to the vehicle power supply through the power supply regulation device 716. In some examples, power is delivered individually to the components of the misting system. Using a power supply regulator can ensure that the misting system is not susceptible to power fluctuations which may damage the misting system. Further, in the example of FIG. 7, controller 702 can be in communication with visual status indicator 720 which can indicate the status of the misting system including its various parts (e.g. misting device 710). For example, visual status indicator can indicate that an element of the misting system is not functioning correctly.

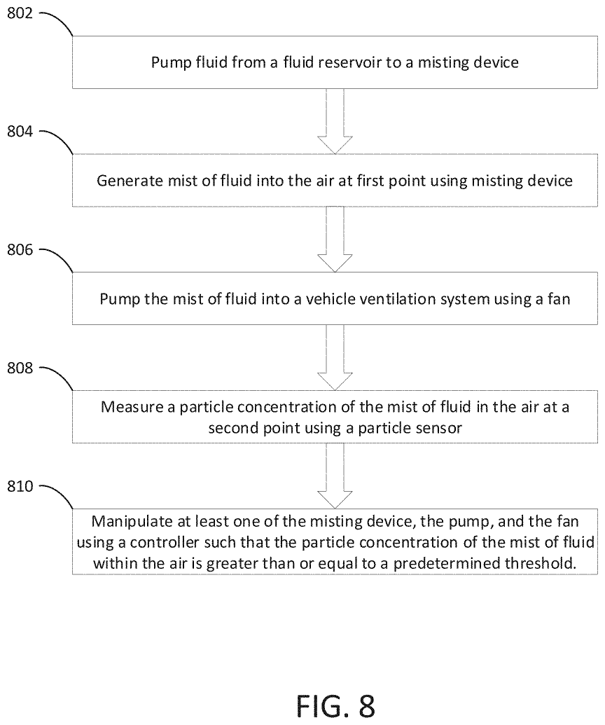

Moving to FIG. 8, FIG. 8 is an example flow diagram of a misting method according to an aspect of the present disclosure. Starting at 802, a fluid is pumped from a fluid reservoir to a misting device using a pump. At 804, a mist of the fluid is generated by the misting device at a first point. At 806, the mist of the fluid is pumped into a vehicle ventilation system using a fan. At 808, a particle sensor measures a particle concentration of the mist of the fluid in the air at a second point. The second point is at a location external to the vehicle ventilation system. At 810, a controller manipulates at least one of the misting device, a heater of the misting device, the pump, or the fan such that the particle concentration of the mist of the fluid in the air is greater than or equal to a threshold.

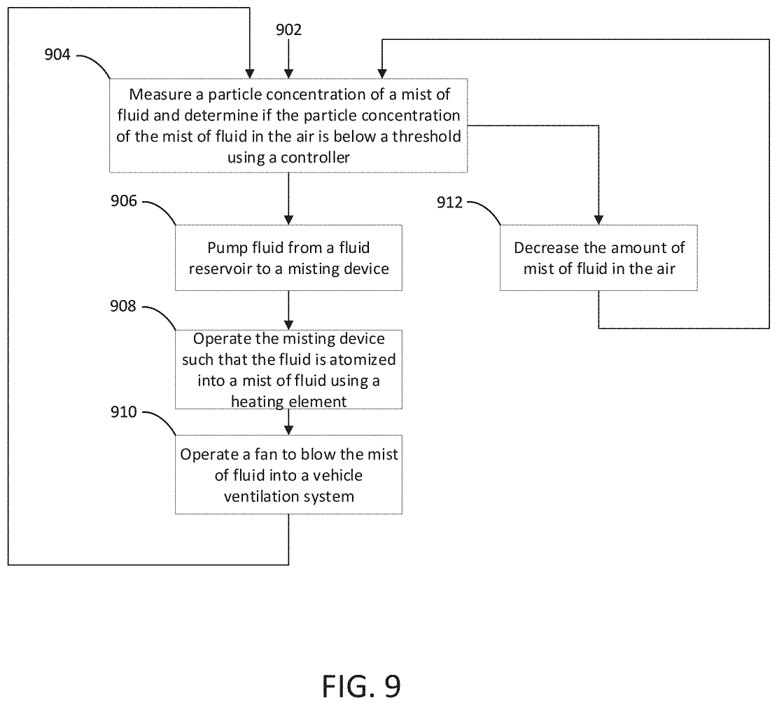

Moving to FIG. 9, FIG. 9 is an example flow diagram of a misting method according to an aspect of the present disclosure. Starting at 902 and moving to 904, a particle sensor measures a particle concentration of a mist of fluid in air and a controller determines if the measured particle concentration is below a threshold. If the measured particle concentration is below the threshold, the method continues with step 906. At 906, fluid is pumped from a fluid reservoir to a misting device using a pump. At 908, the misting device is operated such that the pumped fluid is atomized into a mist of fluid using a heating element. At 910 a fan is operated such that it blows the mist of fluid generated by the misting device into a vehicle ventilation system. The process then repeats with step 904, measuring the particle concentration of the mist of fluid in the air and determining if the particle concentration is below the threshold. However, if the measured particle concentration is at or above the threshold, the method continues with step 912. At 912, the amount of mist of fluid in the air is decreased and then repeats the process at 904. In performing the step of 912, the controller can communicate with the pump, misting device, heating element of the misting device, and/or the fan to decrease the amount of mist of fluid in the air. In some examples, the controller can turn the missing device on and off to decrease the amount of mist of fluid in the air. In some examples, the amount of mist of fluid in the air is kept substantially the same and is not decreased.

While examples of a system and method for generating and controlling mist in a vehicle have been described, the system and method for generating and controlling mist can also be used within enclosed spaces other than vehicles (e.g. a room). For example, a system and method for generating and controlling mist in a room can be provided. In one such example, mist is generated and controlled by a misting system in a similar manner as that described above relative to a vehicle. The misting system used with enclosed spaces can include substantially all the elements of the misting system used with the vehicle. In some examples, the misting system used with enclosed spaces includes a ventilation system which can disperse the mist in and around the enclosed spaces. In some examples, the misting system used with enclosed spaces includes a misting device configured to atomize an amount of fluid to generate a variable amount of mist in the air. The misting system can further include a supply of fluid, a particle sensor, and a controller which is configured to compare the particle concentration to a threshold and adjust various aspects of the misting system to maintain a desired particle concentration. A person of ordinary skill in the art will recognize that other aspects of the misting system for generating and controlling mist in a vehicle can be used for a misting system for generating and controlling mist in an enclosed space and that the present disclosure is not limited to a misting system for generating and controlling mist in a vehicle.

1. A system for generating and controlling a mist in a vehicle comprising:
a vehicle, the vehicle comprising a ventilation system with outlets distributed within the vehicle;
a supply of a fluid;
a misting device configured to atomize an amount of fluid to generate a variable amount of mist in air, the mist having a first range of particle sizes;
a fan having a variable speed and configured to move the mist through the ventilation system and out of the outlets;
a particle sensor configured to measure a quantity and size of particles in air, the particle sensor being located to measure the quantity and size of particles in the air in the vehicle;
a controller in communication with the particle sensor, the controller in communication with and controlling at least one of the variable amount of mist in the air or the variable speed of the fan;
wherein the controller is configured to:
(i) receive the quantity and size of particles in the air in the vehicle from the particle sensor;
(ii) determine a particle concentration in the air in the vehicle, based on the received quantity and size of particles, of particles in a second range of particle sizes, the second range of particle sizes overlapping with the first range of particle sizes;
(iii) compare the particle concentration to a threshold concentration;
(iv) control at least one of the variable amount of mist in the air or the variable speed of the fan in order to have the particle sensor measure a particle concentration greater than the threshold concentration.

2. The system of claim 1, wherein the mist of the fluid is a disinfectant.

3. The system of claim 2, wherein the mist of the fluid is safe for human inhalation.

4. The system of claim 1, further comprising at least one of a temperature sensor, a humidity sensor, and a barometric pressure sensor connected to the controller, wherein the controller receives an input from the at least one of the temperature sensor, the humidity sensor, and the barometric pressure sensor.

5. The system of claim 1, further comprising a pump, the pump fluidly connected to the supply of the fluid and the misting device.

6. The system of claim 5, further comprising one or more sensors to measure at least one of a reservoir fluid level, a dispensing volume of the fluid, a speed of the fan, and the pump, wherein the one or more sensors provide one or more inputs to the controller, the controller configured to use the one or more inputs to determine an amount to adjust the misting device, the pump, and the fan.

7. The system of claim 1, wherein the mist of the fluid within the air comprises of particles between 0.3 microns and 2.5 microns in diameter.

8. The system of claim 1, wherein the vehicle further comprises a vehicle network and wherein the controller is connected to the vehicle network.

9. The system of claim 8, wherein the vehicle network is configured to allow the system to be monitored and controlled remotely.

10. The system of claim 1, further comprising a second particle sensor configured to measure the quantity and size of atomized particles, the particle sensor being located in a second position to measure the quantity and size of atomized particles in the vehicle.

11. The system of claim 1, wherein the particle sensor is laser particle sensor and measures particles having a diameter of between 0.3 microns and 2.5 microns.

12. The system of claim 1, wherein the misting device comprises a heat exchanger and a pump for atomizing the amount of fluid to generate the variable amount of mist in the air.

13. The system of claim 12, further comprising a sensor to determine a level of the fluid, the sensor in communication with the controller, wherein the controller manipulates the pump to control the amount of fluid delivered to the misting device and the variable amount of mist generated.

14. The system of claim 1, further comprising an air inlet, an air outlet, and a filter, wherein the air inlet provides the air to the ventilation system through the filter and the air outlet exhausts the air and the mist.

15. The system of claim 1, wherein the controller includes non-volatile memory for storing information about the system.

16. The system of claim 1, wherein the controller is further configured to connect to a remote device using a network, the remote device for receiving information about a status of the system.

17. The system of claim 1, wherein the misting device further comprises a heating element, the heating element configured to atomize the amount of fluid.

18. A system for generating and controlling a mist in a vehicle comprising:
a vehicle, the vehicle comprising a ventilation system with outlets distributed within the vehicle;
a supply of a fluid;
a misting device configured to atomize an amount of fluid to generate a variable amount of mist in air;
a fan having a variable speed and configured to move the mist through the ventilation system and out of the outlets;
a particle sensor configured to measure a concentration of atomized particles within two or more size ranges of particles, the particle sensor being located to measure the concentration of atomized particles within two or more size ranges of particles in the vehicle;
a controller in communication with the particle sensor, the controller in communication with and controlling at least one of the variable amount of mist in the air or the variable speed of the fan; wherein
the controller is configured to:
(i) receive the concentration of atomized particles within two or more size ranges of particles from the particle sensor;
(ii) determine a concentration of atomized particles having sizes within at least one of the two or more size ranges of particles;
(iii) compare the concentration of atomized particles to a threshold concentration;
(iv) control at least one of the variable amount of mist in the air or the variable speed of the fan in order to have the particle sensor measure a concentration greater than the threshold concentration.

19. A method for generating and controlling a mist in a vehicle comprising:
pumping a fluid from a fluid reservoir to a misting device using a pump;
generating a mist of the fluid at a first point using the misting device such that the mist of the fluid is generated within air;
pumping the mist of the fluid into a vehicle ventilation system using a fan;
measuring a particle concentration of the mist of the fluid within the air at a second point using a particle sensor, the second point being external to the vehicle ventilation system;
manipulating at least one of the misting device, the pump, or the fan using a controller such that the particle concentration of the mist of the fluid within the air is greater than a threshold.

20. The method of claim 19, wherein the mist is a disinfectant and safe for human inhalation.

21. The method of claim 19, wherein the misting device comprises a heating element which atomizes the fluid into the mist of the fluid in the air, the heating element able to be adjusted to change an amount of the fluid atomized by the misting device.
